(19) United States
(12) Patent Application Publication
(10) Pub. No.: US 2003/0088145 A1
(43) Pub. Date: May 8, 2003

(54) Methods and devices for staged thermal and radiation therapy

(76) Inventor: William Tate Scott, San Diego, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/200,009

(22) Filed: Jul. 18, 2002

Related U.S. Application Data
(63) Continuation-in-part of application No. 09/908,475, filed on Jul. 18, 2001, now Pat. No. 6,497,647.
(60) Provisional application No. 60/306,701, filed on Jul. 20, 2001.
(60) Provisional application No. 60/378,611, filed on May 7, 2002.

(51) Int. Cl. A61N 5/00

(52) U.S. Cl. 600/8

(57) ABSTRACT

A method of treating a patient by positioning an implant within a patient, delivering a first therapeutic modality from the implant to the patient, and activating the implant to deliver a second therapeutic modality to the patient, such as by exposing the implant to a magnetic field, is provided. The implant preferably includes a ferromagnetic core, such as a palladium-cobalt alloy. The implant may also include an isotope layer, and an outer layer substantially covering the isotope layer. In one application, the implant enables thermal ablation following unsuccessful brachytherapy, such as in the prostate.

This is a continuation-in-part application of U.S. patent application Ser. No. 09/908,475 filed Jul. 18, 2001, and also claims priority under 35 U.S.C. §119 to U.S. Provisional Application Serial No. 60/306,701 filed on Jul. 20, 2001 and U.S. Provisional Application Serial No. 60/378,611 filed May 7, 2002 the disclosures of which are incorporated in their entirety herein by reference.

### BACKGROUND OF THE INVENTION

1. Field of the Invention

This invention relates generally to the treatment of tissue such as malignant tumors, and, more specifically, to seeds that are implantable into tumorous tissue for simultaneous and/or sequential application of at least thermal energy and radioactive emissions to such tissue.

2. Description of the Related Art

In a journal article entitled “Practical Aspects of Ferro-magnetic Thermoseed Hyperthermia,” published in the Radiologic Clinic of North America, Vol. 27, No. 3, dated May 1989, Ivan A. Brezovich and Ruby F. Meredith, both with the University of Alabama at Birmingham, presented a general treatise on a method of treating tumors by interstitially implanting small pieces of ferromagnetic alloy wire into the tissue and then exposing the subject to an externally applied, oscillating, magnetic field of a predetermined frequency and field strength so as to cause inductive heating of the thermoseeds within the body. This paper points out that by selecting a ferromagnetic material having a suitable Curie point, such a thermoseed becomes self-regulating when the temperature of the seed approaches the Curie point, at which temperature the material becomes non-magnetic. The Carter U.S. Pat. No. 5,133,710 relates to the same technology.

U.S. Pat. No. 5,429,583 to Paulus, et al., which is assigned to the assignee of the present application, describes the use of a palladium-cobalt (Pd—Co) alloy as an improved material for such thermoseeds. By properly adjusting the percent by weight of Pd and Co in the alloy, a Curie point temperature (between 40C and 100C) can be chosen that lies within a range of therapeutic temperatures. Upon exposure to an oscillating magnetic field, the temperature of the thermoseed is self-regulating. The temperature increases until the Curie temperature is reached, at which point, the material becomes non-magnetic, and no additional heating occurs.

It is also known in the art that seeds to be implanted in tumorous tissue can be coated or otherwise treated so as to emit ionizing radiation effective in killing cancerous tissue without excessive damage to surrounding healthy tissue. In this regard, reference is made to Kubiatowicz U.S. Pat. No. 4,323,055, Russell, Jr. et al. U.S. Pat. Nos. 4,702,228 and 4,784,116, Suthanthiran U.S. Pat. No. 4,891,165 and Carden Jr. U.S. Pat. No. 5,405,309, each of which describes techniques for making and utilizing radioactive seed implants and are incorporated by reference herein.

For more than a decade, medical investigators have discussed the synergy of hyperthermia and ionizing radiation in the treatment of several types of tumors. The synergism is believed to be due to some form of combined damage on a cellular level, but increasingly, investigators are theorizing that the increase in blood flow during hyperthermia facilitates the radiation dose by lowering the percentage of hypoxic cells in the tumor. It has been widely known that poorly oxygenated tumors are much more resistant to ionizing radiation than normally oxygenated cell populations. Before the patents cited above, no one appears to have disclosed a combination implant that could produce both thermal and ionizing radiations simultaneously.

Notwithstanding the foregoing advances, there remains a need for a combination therapy device which is capable of delivering radiation, heat and/or other therapeutic modalities to a treatment site.

### SUMMARY OF THE INVENTION

The present invention provides a method of treating a patient, comprising positioning an implant within a patient, delivering a first therapeutic modality from the implant to the patient, and subsequently activating the implant to deliver a second therapeutic modality to the patient. The implant may comprise an implantable seed, wire, endoluminal prosthesis or a stent.

The first therapeutic modality may be delivered by delivering radiation to the patient. The first therapeutic modality delivery is accomplished over a delivery period. The first therapeutic modality delivery may alternatively comprise delivering a drug to the patient. The drug may comprise an anti inflammatory agent, an anti proliferative agent, or an antibiotic.

The implant may be activated by exposing the implant to a magnetic field. A delay is preferably provided between the delivery period and the activation of the implant. The delay may be at least three months or in some applications at least six months.

Another aspect of the present invention provides a two stage method of treating tissue, comprising positioning an implant into tissue to be treated, delivering a dose of radiation from the implant to the tissue, exposing the implant to a magnetic field, and delivering heat to the tissue in response to the exposing step. The exposure to the magnetic field is accomplished following the end of the radiation delivery. Exposure to the magnetic field may be performed at least five days and in some applications at least 3 months or at least 6 months after the end of the delivery of a dose of radiation. The exposing step comprises exposing the implant to an oscillating magnetic field. Preferably, the field has a maximum flux density between about 25 gauss and about 100 gauss, and a frequency between about 25 kHz and 200 kHz.

Another aspect of the present invention provides a method of treating a patient, comprising identifying a patient having a previously positioned implant therein, and activating the implant to deliver a therapeutic modality to the patient. The method may also include clinically assessing the condition of the patient in the vicinity of the implant prior to the activating step.

The implant comprises a ferromagnetic core, such as a palladium-cobalt alloy, whose Curie point temperature is in a therapeutic range, preferably between about 41.5 C and about 100 C. The implant may comprise a decay product of an isotope layer, which may comprise Pd-103. The isotope preferably covers at least about 60%, 85%, or 95% or more of the outside surface of the core. An outer layer may be provided over the isotope layer. The outer layer may have a thickness from about 0.1 micron to about 20 microns. The outer layer may comprise a polymer, metal, which, in one embodiment, is palladium.

The identifying step comprises identifying an implant having a fully decayed isotope thereon. The activating step causes the seed to heat to a temperature within the range of about 40C to about 100C. The activating step preferably comprises exposing the implant to a magnetic field, having a maximum flux density is preferably between about 25 gauss and about 100 gauss and a frequency between about 25 kHz and 200 kHz. The implant may be exposed to an oscillating magnetic field in a plurality of sessions such as at least 2 or 3 or 4 or 5 or more sessions over a course of treatment. By activating the implant, the implant delivers heat to the patient.

In accordance with a further aspect of the present invention, there is provided a method of heating tissue to at least two distinct temperatures from a single source. The method comprises the steps of identifying a medical device in contact with tissue, the device comprising a ferromagnetic material. The tissue is heated to a first temperature in response to exposing the device to a magnetic field under a first set of conditions. The tissue is thereafter heated to a second temperature, in response to exposing the device to a magnetic field under a second set of conditions. The heating the tissue to a second temperature step may be accomplished following an opportunity to clinically assess the efficacy of the first temperature step.

In one implementation of the invention, the first temperature is a hyperthermia temperature. The second temperature may be an ablative temperature. In one application of the invention, the first temperature is a hyperthermia temperature, and the method additionally comprises the step of expressing radiation from the device to the tissue while the tissue is at a hyperthermia temperature.

At least one of the first and second temperatures may be approximately the Curie point for the ferromagnetic material. The other of the first and second temperatures may be below the Curie point for the ferromagnetic material. In one implementation of the method, the other of the first and second temperatures is at least about five and often at least ten or more degrees below the Curie point for the ferromagnetic material. The other of the first and second temperatures, which is the lower of the two temperatures, is achieved by pulsing the magnetic field.

In accordance with a further aspect of the present invention, there is provided a method of heating a medical implant to a temperature below the Curie point for a ferromagnetic material carried by the implant. The method comprises the steps of exposing the implant to an oscillating magnetic field, and pulsing the magnetic field to cooperate with the dissipation of heat from the implant to maintain the implant at a temperature that is below the Curie point.

In accordance with a further aspect of the present invention, there is provided a method of achieving a preselected temperature in tissue surrounding an implanted ferromagnetic material having a Curie point. The method comprises the steps of exposing the material to an oscillating magnetic field, and pulsing the field to achieve the desired temperature in the tissue, wherein the desired temperature is below the Curie point.

In accordance with another aspect of the present invention, there is provided a method of treating a patient. The method comprises the steps of exposing the patient to a magnetic field to generate temperatures in the hyperthermia range at a treatment site within the patient. The patient is thereafter exposed to a magnetic field to generate temperatures in the ablative range at the treatment site within the patient. The method may additionally comprise the step of delivering radiation to the treatment site while the treatment site is at a temperature in the hyperthermia range.

Further features and advantages of the present invention will become apparent to those of ordinary skill in the art in view of the detailed description of preferred embodiments below, when considered together with the attached drawings and claims.

### BRIEF DESCRIPTION OF THE DRAWINGS

FIG. 1A shows an exploded perspective view of an implantable seed for treating cancerous tissue that has one rod-shaped element and two end caps.

FIG. 1B is a cross section of an end cap from FIG. 1A, having a radioactive pellet therein.

FIG. 1C is a cross sectional view of the implantable seed of FIG. 1A, fully assembled.

FIG. 1D is a cross sectional view of the implantable seed of FIG. 1C with both spacers and radioactive pellets in the end caps.

FIG. 2A shows an exploded fragmentary view of a portion of a multi-element implantable seed for treating cancerous tissue wherein adjacent elements are joined together by tubular sleeves.

FIG. 2B is a cross sectional view of a tubular sleeve from FIG. 2A, having a radioactive pellet therein.

FIG. 2C is a cross sectional view of a portion of the multi-element implantable seed of FIG. 2A, fully assembled.

FIG. 2D is a cross sectional view of a portion of the multi-element implantable seed of FIG. 2C with both spacers and radioactive pellets in the tubular sleeves.

FIG. 3A is an exploded perspective view of an alternate implantable device in accordance with the present invention.

FIG. 3B is a transverse cross section through an assembled implantable device of the type illustrated in FIG. 3A.

FIG. 4 is a cross sectional view of an implantable seed for treating cancerous tissue that has a rod-shaped core, a radioactive isotope layer and an outer layer, according to an illustrated embodiment of the current invention.

FIG. 5 is a cross sectional view of an implantable seed for treating cancerous tissue that has a rod-shaped core, a segmented radioactive isotope layer and an outer layer, according to an illustrated embodiment of the current invention.

### DETAILED DESCRIPTION OF THE PREFERRED EMBODIMENT

The present invention is related to the subject matter of previously issued U.S. Pat. Nos. 6,074,337, 5,976,067 and 5,429,583, the disclosures of which are incorporated in their entireties herein by reference.

In general, the present invention provides the combination of a radiation source with a material having a Curie point within a therapeutic range. Certain ferromagnetic materials exhibit a suitable Curie point, either alone or as alloyed with other materials, as will be understood in the art. Ferromagnetic materials useful in this role include iron, cobalt, nickel and manganese. Certain ceramic materials also have a Curie point within the appropriate range, but may not generate sufficient heat for therapeutic purposes. Selection of specific materials can be accomplished through routine experimentation by those of skill in the art, taking into account the desired energy output, the desired characteristics of the externally applied, oscillating magnetic field and the size of the device.

It is believed that there are two distinct mechanisms of treatment in two distinct temperature ranges. The range from 42C to 46C is known as the hyperthermia range wherein tissue is heated above normal temperature and is therefore more susceptible to radiation without necessarily suffering any damage from the heat itself. The range from 46C to 100C is the ablation range wherein tissue is damaged or destroyed from the heat itself. In general, a lower radiation dose can be used in the ablation range than in the hyperthermia range with good therapeutic results.

Preferably, the ferromagnetic material exhibits a Curie point within the range of from about 40C to about 100C, and, in a hyperthermia device, generally within the range from about 40C to about 46C. Certain specific embodiments have a Curie point at about 42.5C or 41.5C.

In addition, the material also desirably has sufficient power output to elevate local temperatures to the above recited temperature values. Sufficient power output generally means greater than about 100 milliwatts per centimeter of length of the seed or rod. Power outputs in excess of about 150 or 200 milliwatts per centimeter length are often preferred, and power outputs of from about 250 milliwatts per centimeters to about 350 milliwatts per centimeter may also be used.

The radiation source component of the present invention can comprise any of a variety of isotopes for emitting gamma, beta or x rays. The radiation source can be in the form of a solid material for entrapment within the cavities as will be discussed below, or in the form of powders, layers or coatings, ion implantation, or other forms depending upon the desired activity, clinical performance and manufacturing techniques. In view of the foregoing, it should be appreciated by those of skill in the art that the following description is exemplary only, and that many variations of the technology described specifically herein will become apparent to those of skill in the art in view of the disclosure herein.

Radioactive material used for radiation treatment is preferably encapsulated to prevent escape of potentially toxic nuclear decay daughter products. For example, an originally inert coating on an implant might be biocompatible radioactive gold. The nuclear decay daughter product of Au¹⁹⁸, however, is mercury, hardly a biocompatible and implantable element. Hence, a combination implant, involving Curie point heating and radioactive dosimetry, preferably has an inert and non-radioactive encapsulation or coating to provide a sealed source and prevent the isotope and/or potentially toxic nuclear decay daughter products from being released into surrounding tissue.

Current encapsulation designs, such as reflected in the prior art references cited herein, are not directly convertible into useable thermoseeds for several reasons. First, the space available within the seed is generally too small to contain a sufficiently large amount of low Curie temperature ferromagnetic core material. If the core material is too small, it cannot produce enough heating power when exposed to an oscillating magnetic field to thoroughly elevate the temperature of surrounding tissue. Second, the length of the space within the seed is too short, such that demagnetization end effects predominate and further reduce the efficiency of the thermoseed. To create a combination seed capable of both self-regulated heating and an adequate radiation dose, known prior art devices must be significantly modified.

In the illustrated embodiments that follow, an implantable seed or therapeutic device capable of producing adequate thermal and ionizing radiation doses for treatment of tumorous or other target tissue is discussed. The implantable seed of the illustrated embodiment is cylindrical, but it should be understood that the seed can alternatively have any of a variety of cross sectional configurations such as tubular, triangular, square, pentagonal or other polygonal, elliptical, lenticular, or any other shape that is suitable for injection into soft tissue.

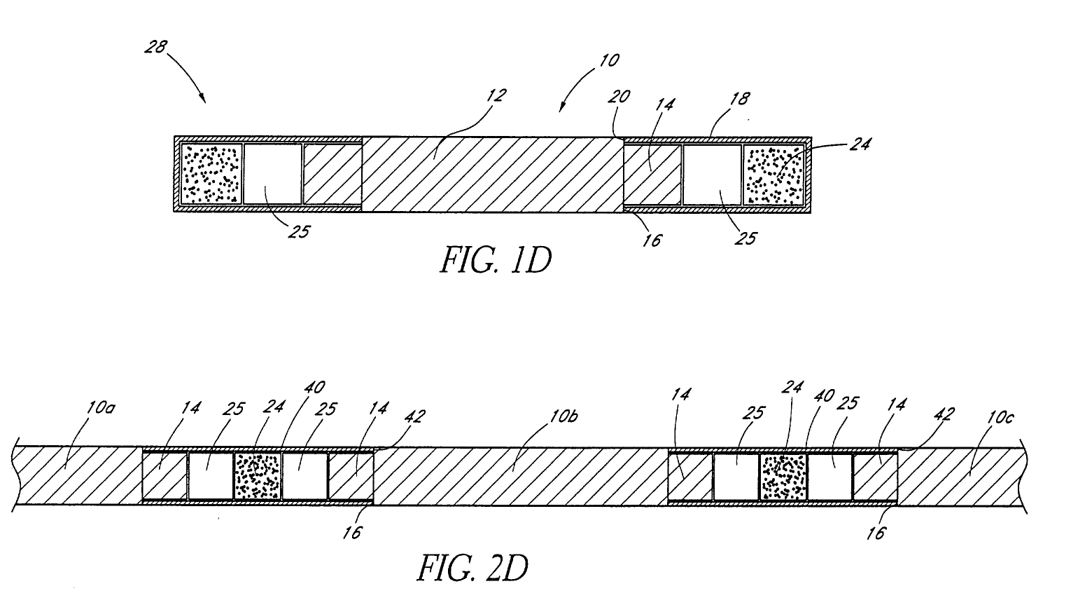

In addition, although described as a “seed” in the present description, persons of skill in the art will notice that certain embodiments, particularly those of FIGS. 2A through 2D, disclose devices which can have a significant length. Thus, the term seed is not intended to convey any kind of aspect ratio or maximum length. Rather, the length of the seeds of the present invention is dictated solely by the desired clinical performance and target tissue.

In addition, although the heat and radiation source aspects of the present invention are disclosed in terms of an implantable seed, the structures and features of the present invention can be readily incorporated into other devices. For example, the ferromagnetic material and radioactive source combinations of the present invention can be readily provided on a portion of an elongate probe such as a sharpened rod or needle, having a handle or control on a proximal end. In use, the needle is advanced percutaneously to the treatment site, with the proximal end remaining outside the patient. Following treatment, the needle may be removed from the patient. In addition, the thermal and radiation delivery structures of the present invention can readily be mounted on the distal end of elongate flexible catheter bodies, such as may be percutaneously or otherwise introduced into the femoral artery, brachial artery or other access point and navigated transluminally through the cardiovascular system to a treatment site.

In addition, the basic support-isotope-coating structure of the present invention may be provided on any of a variety of permanent or temporary implants, such as balloon expandable or self-expandable stents, endoluminal prostheses, soft tissue implants, orthopedic hardware such as bone screws, plates, intermedulary nails, prosthetic femoral stems, and the like, for use in any environment where the delivery of radiation and/or heat may be clinically desirable.

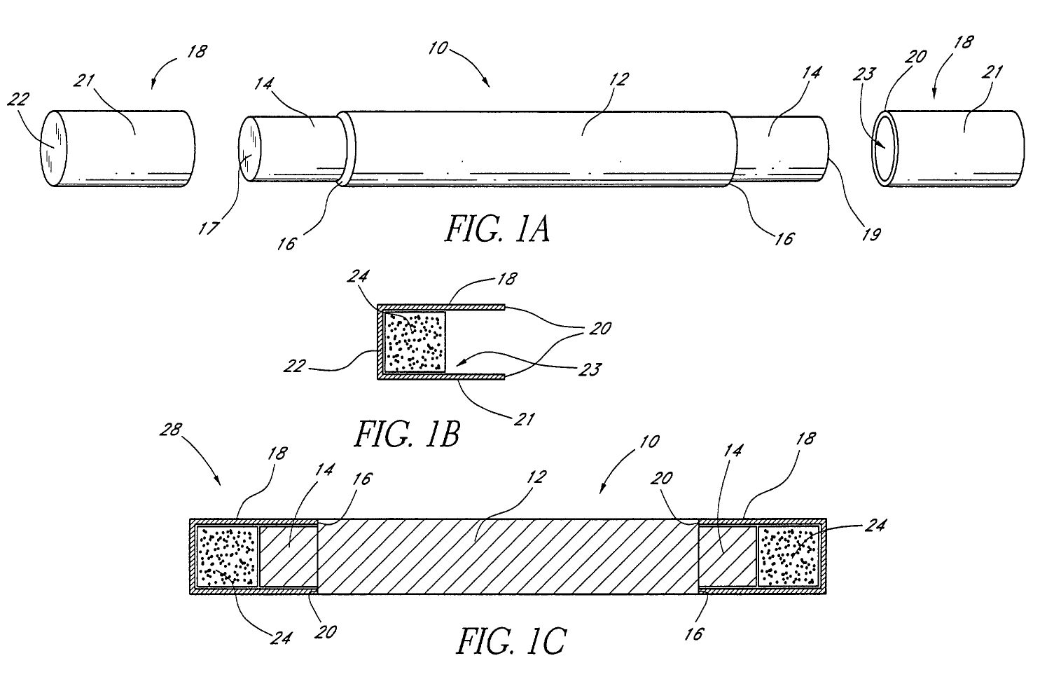

With reference to FIG. 1A, a rod 10, made of a ferromagnetic material, such as nickel-copper, iron-platinum, nickel-silicon, nickel-palladium or palladium-cobalt, with a Curie point temperature between about 40C and 100C., whose middle section 12 is cylindrical in shape, is shown. For application in the prostate, the diameter of the middle section 12 is generally between about 0.8 mm and about 1.2 mm.

The rod end sections 14 of the rod 10 have a smaller diameter than the middle section 12, and the smaller diameter is preferably between about 0.68 mm and 1.20 mm. The length of each rod end section 14 is preferably between about 0.8 mm and 1.2 mm. There are steps 16, perpendicular to the rod surface, that make the transition from the thicker middle section 12 to the rod end sections 14 of the rod 10. The overall length of the rod 10, from a first end surface 17 to a second end surface 19, including the middle section 12 and both rod end sections 14 is between about 6 mm and 14 mm. Preferably the rod is solid from end to end and can deliver power in excess of 150 mw/cm (milliwatts/centimeter), more preferably between about 250 and 350 mw/cm along its length when subjected to an oscillating magnetic field.

End caps 18 are sized to fit over the rod end sections 14 of the Pd—Co rod. Preferably, the end caps 18 comprise a cylindrical side wall 21, but they may have any other configuration that is suitable for attachment to the Pd—Co rod so that the overall shape of the seed is suitable for injection into soft tissue. The end caps 18 each have one open end 20 and one closed end 22, and at least one cavity 23 therein. The outer diameter of the end caps 18 at the open ends 20 may be the same as the outer diameter of the middle section 12 of the Pd—Co rod 10 at the steps 16, to provide a substantially uniform external profile along the length of the seed.

The depth or length of the cavity 23 in the axial direction is greater than the axial length of the rod end section 14, thereby preserving a cavity 23 in the assembled device. Preferably, the depth of the cavity 23 in the axial direction exceeds the axial length of the rod end section 14 by a sufficient distance to accommodate a radioactive source capable of delivering an absorbed dose of at least about 115 to 160 gray over its useable lifetime. Of course, the absorbed dose desired for a specific treatment situation may be different, and a wide variety of radioactive sources and desired doses can be accommodated in the present invention. The volume of the cavity 23 may be varied, depending upon the nature of the source. In an embodiment having a rod end section 14 within the range of from about 0.8 mm to about 1.8 mm in length, the length of the end cap 18 will preferably be at least about 1 mm and generally from about 2 mm to about 5 mm in the axial direction. The depth of the cavity 23, of course, will be only slightly less than the length of the end cap 18. In one embodiment, in which the length of the rod end section 14 is about 1 mm, and the source is palladium-103, the depth of the cavity 23 in end cap 18 is about 2.5 mm. In general, the length of the rod end section 14 will be approximately equal to the desired overlap length between the rod 10 and the end cap 18 in the assembled device. The optimal overlap can be determined through the exercise of routine skill in the art in view of the manner in which the cap 18 is secured to the rod 10, as will be discussed below.

FIG. 1B shows a thin cross section of the end cap 18, into which a radioactive pellet 24 has been placed. The illustrated radioactive pellet 24 is shaped to conform to the cavity 23 inside of the end cap 18. Preferably, the pellet 24 fits snugly against the closed end 22 of the end cap 18, and leaves little or no space between the outer circumference of the pellet 24 and the inner circumference of the end cap 18. When the pellet 24 is in place in the end cap 18, there is at least enough space remaining at the open end 20 of the end cap 18 for making a sufficient connection to the rod 10 (FIG. 1C).

The radioactive pellet 24 can comprise any of a variety of isotopes, depending upon the desired delivered dose, penetration depth into the tissue, and other clinical performance and product shelf life, parameters. In addition, for low energy isotopes, the composition of the cap 18 may limit isotope choice as will be apparent to those of skill in the art. For example, beta emitters (such as phosphorus-32) have relatively low penetration. Certain higher energy sources such as gamma emitters or x-ray emitters have greater tissue penetration but introduce additional complexity during manufacturing and handling. Higher energy sources which may be useful in the context of the present invention include gold-198 (Au¹⁹⁸), iodine-125 (I¹²⁵) and palladium-103 (Pd¹⁰³). Preferably the radioactive pellet 24 comprises Pd¹⁰³ or I¹²⁵. Blends of the foregoing, or other isotopes, may also be used.

The source strength of a radioactive source is related to the number of radioactive events or particles emitted per unit time interval. Given two samples of material with identical half-lives, where one has twice the mass of the other, the larger sample will also have a source strength twice as large. The radiation dose delivered to surrounding tissue is proportional to the source strength of the radioactive emitter.

Given two sources of equal material with different half-lives, initially the source with the shorter half-life will have a greater source strength. Eventually its activity level will fall below that of the other source as the amount of radioactive material in the first source will be depleted faster. Suitable radioactive implants should be capable of delivering more than about 115 gray (joules/kg absorbed radiation dose) and in some embodiments at least about 160 gray over their usable lifetimes. Thus, in designing a radioactive implant, both the half-life and the source strength are important considerations. The half-life is determined completely by the type of radioisotope, and the source strength is determined by both the particular isotope and the amount of radioactive material present. The half-life of Pd¹⁰³ is 17 days, and the half-life of I¹²⁵ is 60 days.

The decay particle energy of the radioisotope is completely unrelated to its half-life or source strength. Typically the decay particle originates from a specific atomic or nuclear event which, in turn, causes the release of x rays of characteristic energy. For example, both Pd¹⁰³ and I¹²⁵ isotopes decay by electron capture, wherein an inner shell electron is absorbed by the nucleus. An outer shell electron jumps down to fill the inner shell vacancy, releasing its excess energy by emitting a characteristic x ray. Due to small variations in the electron energies, characteristic x-ray energies typically fall over a small range. For Pd¹⁰³, these x rays have energies from 20 to 23 keV; for I¹²⁵ these x rays have energies from 25 to 32 keV.

The end cap 18 is made preferably of a material that is biocompatible and that efficiently transmits x rays or other selected decay particles. The end cap material and wall thickness are chosen to allow good transmission of ionizing radiation from the radioactive pellet inside the cap to the surrounding tissue. In one embodiment, the end cap 18 is made of titanium (Ti). The thickness of the Ti end wall 22 and side wall 21 in the end cap 18 are preferably between about 0.02 mm and 0.13 mm.

FIG. 1C is a side elevational cross-sectional view through a fully-assembled two-source implantable seed according to one embodiment of the current invention. The inner circumference of the end caps 18 fits snugly over the outer circumference of the rod end sections 14 of a Pd—Co rod 10. The open ends 20 of the end caps 18 fit snugly against the steps 16 of the Pd—Co rod. The outer diameter of the end caps 18 and the outer diameter of the middle section 12 of the Pd—Co rod 10 are substantially the same at this junction, as discussed above for FIG. 1A, making the outer surface of the implantable seed 28 smooth and continuous throughout.

The end cap 18 may be connected to the rod 10 in any of a variety of manners, as will be apparent to those of skill in the art in view of the disclosure herein. In general, the connection between the end cap 18 and rod 10 will take into account the respective materials of these two components, together with the desired integrity of the bond. For example, for metal end caps 18 and rods 10, any of a variety of welding, soldering, brazing or other metal bonding techniques may be used. Interference fit, such as snap fit constructions may also be used. Complementary surface structures such as a male thread on the end section 14 for cooperation with a corresponding female thread on the end cap 18 may also be used. Outer polymeric or metal coatings to span across the rod 10 and end caps 18 may also be used.

Preferably, the bonding technique will both provide sufficient physical integrity to prevent detachment of the cap 18 during normal use conditions, as well as enable the finished seed to qualify as a sealed radioactive source. In an embodiment having a titanium end cap 18 and a Pd—Co rod 10, the end cap 18 may be welded to the rod 10. In other embodiments, such as toleranced or interference fit structures, additional bonding agents such as adhesives or other polymeric materials may be utilized to assist in meeting the sealed radioactive source standard. If a polymeric species is utilized as a bonding agent or sealing agent, interactions should first be determined between the particular polymeric species and the nature and activity of the isotope, in view of the degradation which can occur to polymeric materials when positioned in a radioactive field.

Alternatively, it may be desirable to position the radioactive source a distance away from the Pd—Co rod in order to decrease attenuation of radiation by the Pd—Co adjacent to the rod. As shown in FIG. 1D, a spacer 25 can be placed between rod end section 14 and radioactive pellet 24 in the end cap 18. Preferably the spacer comprises a material that is a good transmitter of radiation, for example, silica glass, silicon, beryllium or aluminum.

In another embodiment of the current invention, a multi-element implantable seed, wire or probe wherein the ferromagnetic rod comprises at least two separate pieces joined together by tubular sleeves which also hold radioactive pellets that act as point sources, can be understood with reference to FIGS. 2A-2D. This arrangement can accommodate three or four or five or more radioactive point sources arranged spaced apart along the length of the device. The skilled artisan can choose the number of point sources and the distance between the sources to tailor the ionizing radiation dose distribution provided by the device for optimal treatment of the surrounding tissue.

FIG. 2A is an exploded side elevational view of a portion of a multi-element implantable seed that shows ferromagnetic rods 10a, 10b, 10c preferably comprising Pd—Co with a Curie point temperature between about 40C and 100C, as has been described above with reference to FIG. 1A. In this illustration, the middle section 12 of each rod 10 is cylindrical in shape, preferably with a diameter between about 0.8 mm and 1.2 mm. Many of the details of the embodiment of FIGS. 1A-1D discussed supra may be readily applied to the embodiment of FIGS. 2A-D, which will be discussed only briefly below.

The rod end sections 14 have a smaller diameter than the middle section 12, and the smaller diameter is preferably between about 0.68 mm and 1.20 mm. The length of each rod end section 14 is preferably between about 0.8 mm and 1.2 mm. There are steps 16, which may be perpendicular to the rod surface, that make the transition from the thicker middle section 12 to the thinner rod end sections 14. The overall length of each rod 10a, 10b and 10c, from one end surface 17 to another 19, including the middle section 12 and both rod end sections 14, is between about 6 mm and 14 mm. Preferably the rod is solid from end to end and can deliver power in excess of 150 mw/cm, more preferably, between about 250 and 350 mw/cm, along its length when subjected to an oscillating magnetic field.

The tubular sleeve 40 is a hollow tube with two open sleeve ends 42. The tubular sleeve 40 is at least long enough to accommodate a radioactive pellet 24 and two rod end sections 14 of the adjacent Pd—Co rods 10, one at each sleeve end 42. Preferably the tubular sleeve 40 is between about 3.0 mm and 6.0 mm in length. The outer diameter of the sleeve ends 42 is substantially the same as the outer diameter of the adjacent portions of the middle section 12 of the Pd—Co rod 10 at the steps 16.

FIG. 2B shows a thin cross section of the tubular sleeve 40, into which a radioactive pellet 24 has been placed. The radioactive pellet 24 is shaped to conform to the central portion of the tubular sleeve 40. Preferably, the pellet 24 fits snugly against the wall 44 of the tubular sleeve 40, with little or no space between the outer circumference of the pellet 24 and the inner circumference of the tubular sleeve 40. When the pellet 24 is in place in the tubular sleeve 40, there is at least enough space remaining at each open end 42 of the tubular sleeve 40 for making connections to a Pd—Co rod 10 (FIG. 2A) at each sleeve end 42. Preferably the radioactive pellet 24 comprises palladium-103 (Pd¹⁰³) or iodine-125 (I¹²⁵).

The tubular sleeve 40 is made preferably of a material that is biocompatible and that transmits x rays or other radioactive species well. More preferably, the tubular sleeve 40 is made of titanium. The thickness of the Ti wall 26 in the tubular sleeve 40 is preferably between about 0.025 mm and 0.050 mm. FIG. 2C is a longitudinal cross-sectional view through the middle of a fully-assembled, multi-element implantable seed. The inner circumference of the tubular sleeve 40 fits snugly over the outer circumference of the rod end sections 14 of the Pd—Co rod 10. The open ends 42 of the tubular sleeve 40 fit snugly against the steps 16 of the Pd—Co rod. The outer diameter of the tubular sleeve 40 and the outer diameter of the middle section 12 of the Pd—Co rod 10 are the same at this junction, as discussed above for FIG. 2A, making the outer surface of the implantable seed 28 smooth and continuous throughout. The tubular sleeve ends 42 are connected such as by welding to the Pd—Co rod 10 at the junctions.

The radioactive pellet 24 has a length that fits into the space remaining in the tubular sleeve 40 after the Pd—Co rods 10 have been attached at both ends. Preferably the pellet 24 fits snugly against the end surfaces 17 of the Pd—Co rods 10 on each end of the tubular sleeve 40. The length of the tubular sleeve 40 can be adjusted to accommodate radioactive pellets 24 of various lengths and spacers if desired. The outermost ends of the multi-element implantable seed can be sealed off with end caps as shown in FIGS. 1A-1D.

The length of the Pd—Co rods and any spacers used determines the spacing between radioactive pellets, which act as radiation point sources in the implantable seed. The skilled artisan can choose a spacing and a number of radioactive pellets to provide a desired dose distribution to the soft tissue surrounding the implantable seed. It may be desirable to position the radioactive source a distance away from the Pd—Co rods in order to decrease attenuation of radiation by the Pd—Co adjacent to the source. As shown in FIG. 2D, spacers 25 can be placed between rod end sections 14 and radioactive pellet 24 in the tubular sleeve 40. Preferably the spacers comprise a material that is a good transmitter of radiation, for example, silica glass, silicon, beryllium or aluminum.

Metal tubes with their own structural integrity are not the only means for connecting ferromagnetic rods and enclosing radioactive pellets and spacers. For example, tubes can be made of other materials, such as plastic or glass. Alternative arrangements can also be used. Rods, pellets and spacers can also be held together by films or coatings applied by dipping, spraying or wrapping. Preferably films or coatings are at least several μm in thickness and can be as thick as 10 μms or more.

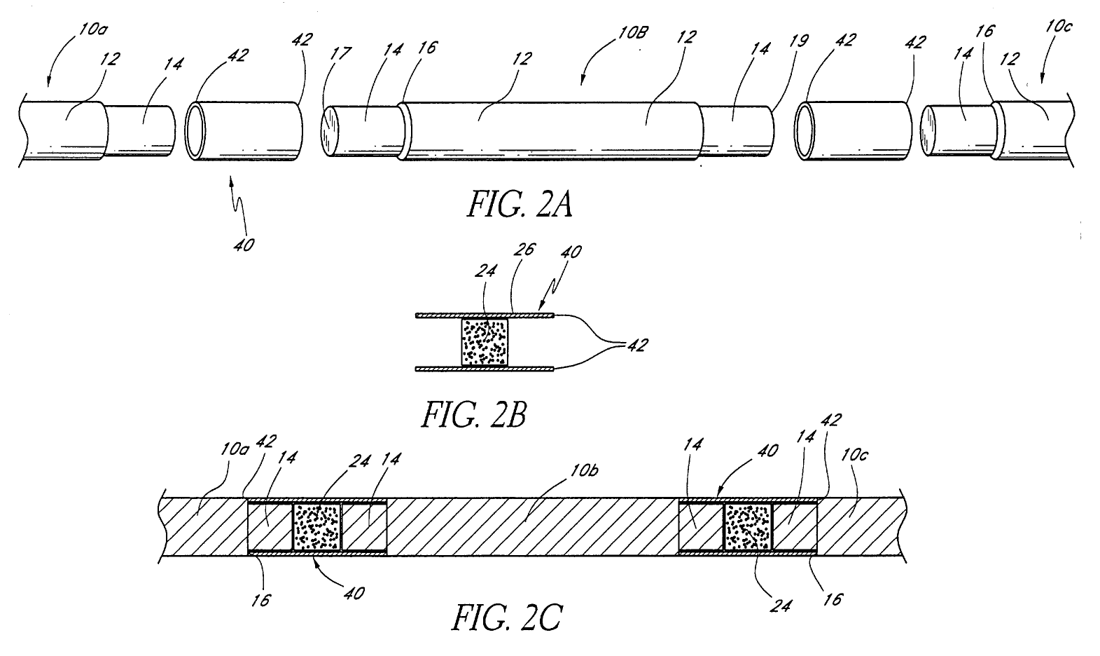

A further implementation of the present invention is illustrated in FIGS. 3A and 3B. In this implementation, any of the materials and dimensions previously discussed may be utilized and will therefore not be repeated in detail below. In this embodiment, a seed 50 comprises a rod 52 having one or more axially-extending channels 54. The channel 54 may be machined, milled, molded, stamped or otherwise created, in accordance with manufacturing techniques which will be well understood by those of skill in the art, and dependent upon the material of the rod 52.

At least one radioactive source 56 is positioned within the channel 54. An outer tubular coating or sleeve 62 is coaxially positioned over the rod 52 both to retain the source(s) 56 within channel(s) 54 and to provide a seal between the source(s) 56 and the outside environment. One manner of accomplishing this seal is to provide the channel 54 with an axial length of less than the axial length of the rod 52. As illustrated in FIG. 3A, this permits a first sealing zone 58 at a first end of the source 56 and a second sealing zone 60 at a second end of the source 56. The sleeve 62 is configured to fit snugly around the rod 52, such that a seal is created at the sealing zones 58 and 60 to provide a seal.

Referring to FIG. 3B, one embodiment of an assembled device is illustrated in cross section. In this embodiment, four sources 56 are positioned on the rod 52, and spaced at 90° intervals. One or two or three or four or more sources 56 may be positioned circumferentially about the rod 52, depending upon the desired activity and delivered dose profile. As the number of sources 56 is increased, the radiation delivery profile of the seed 50 will approach that which would be achieved by a continuous tubular sleeve of radioactive source, concentrically positioned about the rod 52.

The present invention contemplates the use of a concentric construction in which the rod 52 carries a tubular source sleeve (not shown), which is in turn entrapped within an outer sleeve 62. In an embodiment having a cylindrical source, the source preferably resides within an annular channel on the rod 52, such that the outside diameter of the assembled source is approximately equivalent to the outside diameter of the rod 52 in the first and second seal zones 58 and 60. In this embodiment, a cylindrical source may be positioned on a rod 52 having a constant outside diameter, and held in place by positioning a short tubular locking sleeve on one or both ends of the rod in the first and second seal zones 58 and 60, as will be apparent to those of skill in the art in view of the disclosure herein. Thereafter, a constant diameter sleeve 62 may be positioned on the assembly and sealed.

Referring back to FIG. 3B, each of the channels 54 is illustrated as having a generally triangular cross section. Any of a variety of cross-sectional configurations for the channels 54 may be utilized, such as round, square, rectangular or radiused curve, depending upon the desired volume of the source 56 as well as the preferred manufacturing techniques for creating the channel 54.

The outer tubular sleeve 62 may be mounted on the rod 52 in any of a variety of ways, depending upon the construction materials. For example, for a metal sleeve 62 and a metal rod 52 (for example, both made of Pd—Co), the inside diameter of the sleeve 62 may be approximately equal to or slighter smaller than the outside diameter of the rod 52. The rod 52 may be cooled, and/or the sleeve 62 may be heated, to allow coaxial advancement of the sleeve 62 over the assembly of the rod 52 and the sources 56. Additional sealing steps, such as welding, may be accomplished on the axial ends of the seed 50, to ensure the integrity of the bond between the sleeve 62 and the rod 52.

Alternatively, sleeve 62 may comprise any of a variety of polymeric materials which shrink upon application of heat. A variety of heat shrink tubing materials are well understood in the catheter manufacturing arts. As a further alternative, a sleeve 62 may be applied to the rod 52 and radially outwardly facing surfaces of the sources 56 such as by dipping, spraying or wrapping operations.

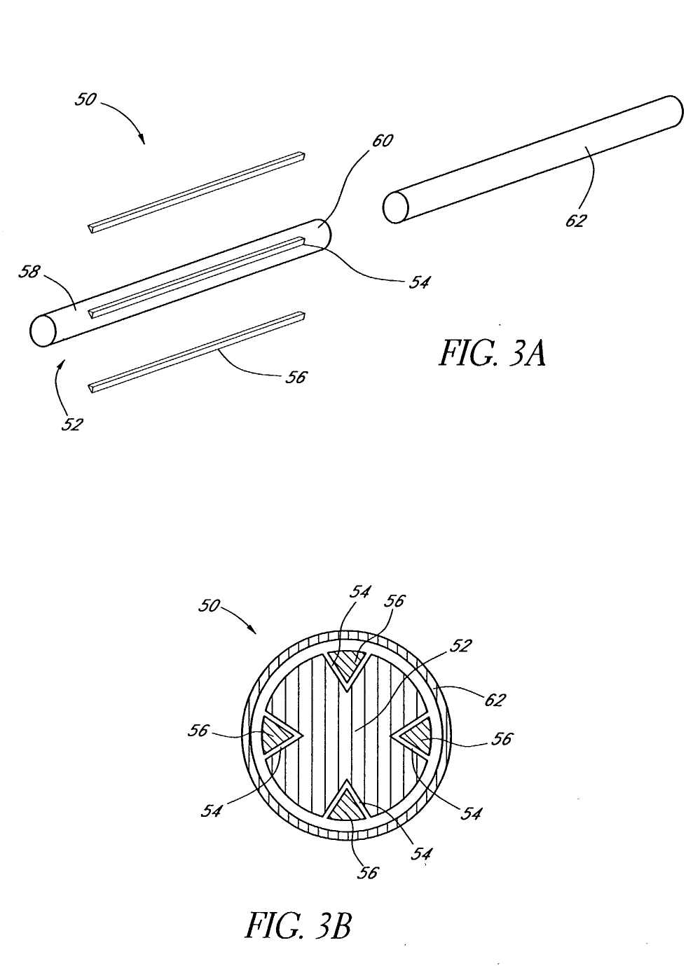

With reference to the illustrated embodiment of FIG. 4, an implantable seed 100 for treatment of cancer is shown. A support such as a rod 120, made of a ferromagnetic material, such as nickel-copper, iron-platinum, nickel-silicon, nickel-palladium or palladium-cobalt, with a Curie point temperature between about 40C and 100C, and having a cylindrical shape, comprises the core of the seed 100. The rod 120 may be tubular or solid from end to end and can deliver power in excess of 150 mw/cm (milliwatts/centimeter), more preferably between about 250 and 350 mw/cm along its length when subjected to an oscillating magnetic field.

The overall length of the seed 100, from one end surface 125 to the other is between about 2 mm and 14 mm. For application in the prostate, the overall diameter of seed 100 is generally between about 0.8 mm and about 1.2 mm, and the length in one embodiment is about 4 mm.

The core rod 120 is coated, at least in part, by a radioactive isotope. Possible coating methods include, but are not limited to, wrapping, dipping, spraying, sputtering, evaporating, electroless plating and electroplating. It is preferable that the radioactive isotope coating or layer 140 forms a strong bond to the core 120. Various isotope bonding technologies are disclosed in, for example, U.S. Pat. Nos. 6,210,313 B1, 6,196,963 B1, 6,192,095 B1, 6,187,037 B1, 6,183,409 B1, 6,163,947, 6,129,658, 6,103,295, 6,077,413, the disclosures of which are hereby incorporated in their entireties herein by reference

Preferably, the choice of isotope and the amount of isotope in the coating 140 produce a radioactive source capable of delivering an absorbed dose to surrounding tissue of at least about 115 to 160 gray over its useable lifetime. Of course, the absorbed dose desired for a specific treatment situation may be different, and a wide variety of radioactive sources and desired doses can be accommodated in the present invention. In one embodiment, the seed is approximately 4 millimeters long, and spaced approximately 1 centimeter apart along the length of a treatment needle. The seeds produce within the range of from about 0.5 to about 3 mCuri per seed, and, in one embodiment, about 1.2 mCuri per seed.

The radioactive coating 140 can comprise any of a variety of isotopes, depending upon the desired delivered dose, penetration depth into the tissue, and other clinical performance and product shelf life parameters. High energy sources, which may be useful in the context of the present invention, include gold-198 (Au198), iodine-125 (I125) and palladium-103 (Pd103). Preferably the radioactive coating 140 comprises Pd103. Blends or alloys of the foregoing or other isotopes may also be used.

In addition, for low energy isotopes, the composition and thickness of the outer sealing layer may limit isotope choice as will be apparent to those of skill in the art. For example, beta emitters (such as phosphorus-32) have relatively low penetration. Certain higher energy sources such as gamma emitters or x-ray emitters have greater tissue penetration but introduce additional complexity during manufacturing and handling.

The isotope layer 140 may completely cover the core rod 120, as shown in the illustrated embodiment in FIG. 4. Preferably, the isotope layer 140 covers at least 60% of the outside surface of the core 120, more preferably, at least 85% and, most preferably, the isotope layer 140 covers at least 95% of the outside surface of the core 120. In one embodiment, the isotope covers substantially the entire surface of the seed, including the ends.

The isotope layer is covered, at least in part, by an outer layer 160. Preferably the isotope layer 140 and the core 120 are encapsulated entirely by outer layer 160. The outer layer 160 comprises a material that is biocompatible and that efficiently transmits x rays or other selected decay particles. The thickness of the outer layer is chosen to allow good transmission of ionizing radiation from the radioactive isotope layer 140 to the surrounding tissue.

In one embodiment, outer layer 160 comprises a metal. Preferably the outer layer 160 comprise Pd with a thickness from about 0.1 micron to about 20 microns. In many embodiments, the outer layer is within the range of from about 1 to about 12 microns thick. In the case of a palladium outer layer, thicknesses in excess of about 12 microns begin to attenuate the magnetic field. Outer layers of appropriate thicknesses can be applied in any of a variety of manners as has been identified herein. In general, electroplating provides a useful inexpensive and controllable manufacturing technology. Electroless plating may also be used. Other metals for use in the outer layer include, but are not limited, to gold, titanium, beryllium and aluminum. Silicon, silica glass or Tekoflex can also be used for outer layer 160. In general, the coating preferably provides a sealed source, to substantially prevent the escape of isotope into the body. In addition, the outer layer preferably does not unduly shield the theromagnetic core or attenuate the penetration of radiation, yet it provides a sufficient physical barrier against abrasion to protect the isotope during handling steps.

In another embodiment, the outer layer 160 comprises a polymer. If a polymeric species is used for the outer layer 160, interactions between the particular polymeric species and the radiation from the isotope layer 140 should first be well understood, in view of the degradation that can occur to polymeric materials with prolonged exposure to radiation.

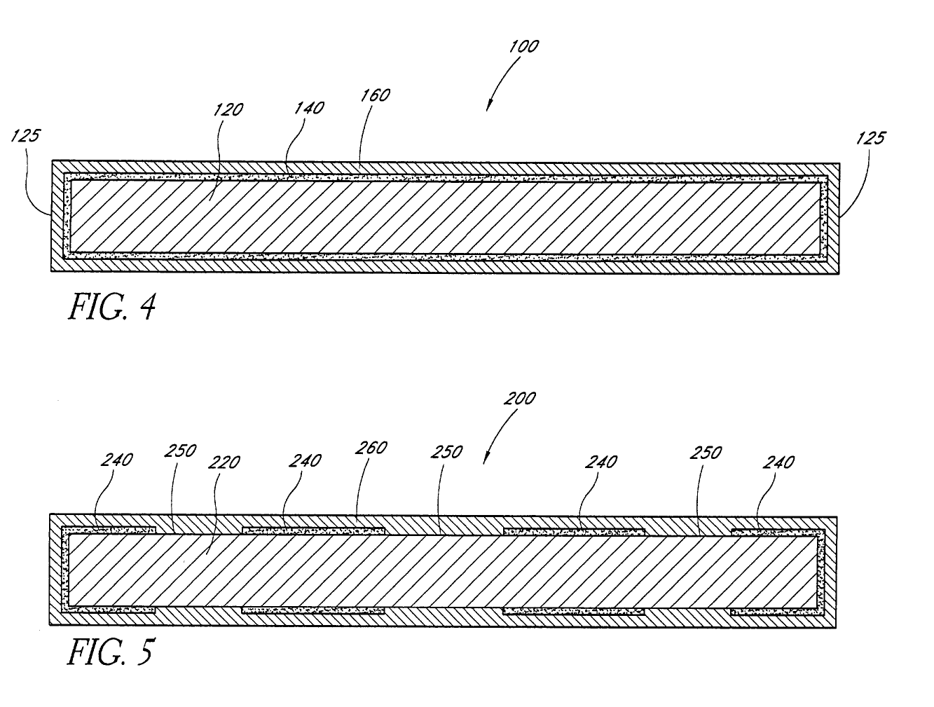

In another illustrated embodiment, FIG. 5 shows a cross section of an implantable seed 200 with a series of radioactive “point” source coating segments 240. As for the embodiment illustrated in FIG. 4, a rod 220, made of a ferromagnetic material, such as nickel-copper, iron-platinum, nickel-silicon, nickel-palladium or palladium-cobalt, with a Curie point temperature between about 40C and 100C, and having a cylindrical shape, comprises the core of the seed 200.

The core rod 220 is coated in discreet sections by a radioactive isotope. Possible coating methods include, but are not limited to, wrapping, dipping, spraying, sputtering, evaporating, electroless plating and electroplating as has been discussed. Regions 250 wherein no radioactive coating is desired can be masked during the coating process by any number of methods. It is preferable that the radioactive isotope coating or layer segments 240 form strong bonds to the core 220. This arrangement can accommodate three or four or five or more radioactive coating segments 240 arranged spaced apart along the length of the rod 220. The skilled artisan can choose the number of point sources and the distance between the sources to tailor the ionizing radiation dose distribution provided by the seed for optimal treatment of the surrounding tissue.

The core rod 220 and radioactive coating segments 240 are covered, at least in part, by an outer layer 260, as has been discussed. Preferably the isotope layer 240 and the core 220 are encapsulated entirely by outer layer 260. The outer layer 260 comprises a material that is biocompatible and that efficiently transmits x rays or other selected decay particles. The thickness of the outer layer is chosen to allow good transmission of ionizing radiation from the radioactive isotope layer 240 on the core 220 to the surrounding tissue. In one embodiment, outer layer 260 comprises a metal. Preferably the outer layer 260 comprise Pd with a thickness from about 0.1 micron to 20 microns. Other metals for use in the outer layer include, but are not limited, to titanium, beryllium and aluminum. Silicon or silica glass can also be used for outer layer 260.

Alternatively, outer layer 260 may comprise any of a variety of polymeric materials such as those which shrink upon application of heat. A variety of heat shrink tubing materials are well understood in the catheter manufacturing arts. As a further alternative, an outer polymer layer 260 may be applied to the rod 220 and radioactive source segments 240 by operations such as dipping, spraying or wrapping.

In another embodiment of the current invention, a method of making an implantable seed for supplying thermal and ionizing radiation to cancerous tissue is provided. The seed comprises rod-shaped ferromagnetic alloy elements and radioactive sources, as described herein.

Compositional precision is necessary to produce a ferromagnetic alloy with a specific Curie point. For example, in Pd—Co alloys, a variance in composition by as little as 0.03%, by weight, changes the Curie point by 1° C. The range of desirable Curie temperatures, 40C to 100C, can be achieved by varying the composition of the Pd—Co alloy by only about 2%, i.e., from about 5.5 wt % to 7.5 wt % cobalt, as shown in Table 1 below.

TABLE 1

| Wt % Cobalt | Curie Temperature |
|---|---|
| 5.75 | 40° C. |
| 6.20 | 55° C. |
| 6.35 | 60° C. |
| 7.55 | 100° C. |

The exemplary palladium-cobalt alloy is produced preferably using an induction melting technique. Palladium and cobalt pellets or powders are placed in a sealed vessel under inert gas and melted using an induction coil. The vessel is pressurized above the vapor pressure of liquid Pd so that vaporization of this more volatile species is minimized. Preferably the vessel is designed so that the resulting Pd—Co ingot is cylindrical in shape.

The alloyed ingot cylinder, typically 6 mm to 12 mm in diameter, is then mechanically swaged and drawn into a rod of desired diameter, preferably 0.8 mm to 1.2 mm. Variations in the composition of the material can occur as the ingot is drawn out to smaller diameters. Thus, it is preferable to begin cold working the alloy only after it has been fully homogenized. High temperature annealing of the alloy slightly below the melting point, at 1000C to 1100C, for a few hours appears to be sufficient to homogenize Pd—Co.

After the rods have been fully drawn and cut into appropriate lengths, they are given one final heat treatment to allow recrystallization and grain growth, as is known in the art. This annealing step can be done in a single zone furnace in an inert gas atmosphere. The rods are then furnace cooled to prevent oxidation.

The implantable seed is assembled with at least one exemplary Pd—Co alloy rod or element. Radioactive sources, preferably in the form of pellets, are positioned adjacent to the ends of the alloy element and are held in place at each end with a cylindrical tube with one closed end, which fits over the end of the element. In alternative embodiments, multiple elements are used to assemble the seed. The elements are held together by cylindrical tubes that fit over the ends of the elements, and radioactive pellets and optional spacers are positioned in the cavities in the tubes between the elements. Preferably the cylindrical tubes are made of a material that transmits radiation, such as titanium, and are sealed to the alloy elements by welding.

An implantable seed or other support may be manufactured with an exemplary Pd—Co alloy rod or core. A layer of radioactive isotope, preferably palladium-103, is coated onto the core and is encapsulated with an outer layer of biocompatible material to provide a sealed-source radioactive seed. In alternative embodiments, the radioactive isotope layer can be applied discontinuously, thereby providing segments, separated along the rod length, that are essentially radiation point sources. Preferably the outer encapsulating layer comprises a material that transmits radiation, such as a polymer or a metal such as palladium, titanium, beryllium or aluminum.

In use, the combination seed or other combination thermal-radiation implant is percutaneously or surgically positioned within or adjacent tissue to be treated within the body. Alternatively, the combo support may be positioned within the body through an external opening thereon, such as transesophageally for the purpose of treating certain esophageal cancers or other conditions. In the case of a solid tumor treatment, typically a plurality of seeds will be stacked into an alternating column, such that seeds are spaced apart along an axis. Then a plurality of the columns of seeds and spacers may be positioned within the soft tissue, generally extending in parallel to one another and spaced apart throughout the tissue. The placement and theory behind placement of radioactive seeds in treatment sites within the body is well understood in the art, and need not be described in greater detail herein.

An external oscillating magnetic field, preferably with a maximum flux density between 25 gauss and 100 gauss and a frequency between 25 kHz and 200 kHz, is supplied, which acts upon the exemplary Pd—Co alloy elements. Pd—Co heats up under the influence of the oscillating magnetic field until the Curie point temperature is reached.

In another embodiment, a method of treating a patient is provided. A plurality of implantable seeds, comprising ferromagnetic or other Curie point material and at least one source as discussed above, is positioned in cancerous tissue such that the longitudinal axes of the seeds are parallel. The Curie point temperature of the ferromagnetic material is in a therapeutic range. The implantable seeds are exposed to an external oscillating magnetic field aligned generally parallel to the longitudinal axes of the seeds. Under the influence of the oscillating magnetic field, the seeds heat to their Curie point temperature. Radioactive sources are positioned as a coating, or in cavities defined, in part, by the end caps that are attached over the ends of the implantable seeds. The radioactive sources provide ionizing radiation to treat the cancerous tissue. The radioactive sources may be, among others, palladium-103 or iodine-125.

Alternatively, each implantable seed can comprise sections of ferromagnetic material connected by hollow, tubular sleeves, in which radioactive sources and optional spacers are positioned. Preferably, the radioactive sources are positioned to produce a uniform dose profile around each implantable seed. The skilled artisan can adjust section lengths and radioactive source sizes to tailor a radiation dose profile for a particular treatment situation.

The implantable seeds can be exposed to an oscillating magnetic field for delivering heat energy to the cancerous tissue in a plurality of sessions over a course of treatment, even after the strength of the radioactive sources has diminished to sub-therapeutic levels.

In another embodiment, another method of treating a patient is provided. An implant, such as a stent, an orthopedic device, solid or soft tissue implant or endoluminal prosthesis as previously described, is positioned within a patient. A first therapeutic modality is delivered from the implant to the patient, such as by delivering a drug or radiation to the patient. The drug may be an anti inflammatory agent, an anti proliferative agent, or an antibiotic. The first modality is preferably delivered over a delivery period. A second therapeutic modality is delivered to the patient by activating the implant. The implant may be activated such as by exposing the implant to a magnetic field. A delay may be provided between delivery of the first and second therapeutic modalities. The delay may be at least about 5 days, or at least about three months, or at least about six months or more and, in any event, following an evaluation of the patient to assess the efficacy of the first modality.

Thus, the combination thermal-brachytherapy implant may be implanted to delivery a therapeutic dose of radiation to a treatment site. Following the delivery period, if the tumor persists or treatment has otherwise not been fully effective, the previously implanted device can be exposed to a magnetic field to generate ablative temperatures and produce a secondary treatment. This allows bail out ablation therapy to be accomplished in a patient if brachytherapy has failed, without the need to position additional probes or implants within the patient.

In accordance with another aspect of the present invention, a two stage method of treating tissue is provided. The tissue is treated by positioning an implant into the tissue to be treated, delivering a dose of radiation from the implant to the tissue, exposing the implant to a magnetic field, and delivering heat to the tissue in response to exposing the tissue to the magnetic field. Preferably, the implant is exposed to the magnetic field after the radiation delivery has been completed. In certain embodiments, the exposure of the implant to a magnetic field occurs at least about 5 days after the end of the delivery of radiation. Preferably, the magnetic field is an oscillating magnetic field, having a maximum flux density between about 25 and 100 gauss and a frequency between about 25 kHz and 200 kHz.

In any of the methods disclosed herein, heat may be applied during at least a part of the radiation delivery step as well as by itself as a follow on step. For example, heat may be applied during at least a portion of the radiation delivery step, to a temperature within the hyperthermia range to increase the efficacy of the radiation therapy. This may be for example within the range of from about 42° C. to about 46° C. Following a period of days or months, the patient may be evaluated to determine the efficacy of the first phase of the treatment. If warranted, the patient may be reexposed to a magnetic field to reheat the implants, such as to an ablation temperature (e.g. from about 46° C. to about 100° C. in certain applications about 70° C.) to produce localized ablation. The secondary step of applying localized ablation temperatures may be desirable where the radiation therapy failed to achieve its clinical objective.

As will be appreciated by those of skill in the art in view of the disclosure herein, any of a variety of combinations of therapy can be utilized in a first stage of treatment, as well as a second stage of treatment, and a third stage or fourth or fifth or more, depending upon the perceived clinical need. Referring to the table below, certain representative staged therapy combinations are illustrated. These illustrated combinations are non-exhaustive, as will be apparent from a review of the table. In this table, R represents radiation therapy, either from an onboard isotope, as has been described previously herein, or from an external source, such as electron beam radiation therapy. H represents the delivery of heat in the hyperthermia range, and A represents the delivery of heat in the ablative temperature range.

Representative Staged Therapy Combinations

| STAGE I | STAGE II | STAGE III |
|---|---|---|
| R ALONE | A or R + H or R + A | Any of R, H, A or |
| H ALONE | A or R or R + H | Combinations, repeated as |
| A ALONE | A or R or R + H or R + A | clinically desired |
| R + H | A or R + A or R + H | |
| R + A | A or R + A or R + H | |

As illustrated, Stage 1 therapy may comprise radiation alone, hyperthermia heat alone, ablative heat alone, or the combination of radiation and hyperthermia or radiation plus ablative heat. In many applications, Stage 1 therapy will either be radiation alone, or radiation delivered simultaneously with hyperthermia heat. However, other therapies may be utilized. In addition, any of the therapies represented in the table may be combined with drug delivery, either from the implant itself, or from a remote site. Medication may either be configured to release upon the application of heat, or be attracted from a remote delivery site to a source of heat in the body.

Depending upon the clinical results of the Stage 1 therapy, the clinician may determine that a Stage 2 therapy is desirable. Representative but non-exhaustive Stage 2 therapies are identified in the table. As one example, if radiation alone, or radiation plus hyperthermia heat, or radiation plus drug, or radiation plus hyperthermia heat plus drug was accomplished in the Stage 1 therapy, and if the desired clinical result was not achieved, the clinical objective may be to attempt a more aggressive treatment, such as ablation. Thus, the Stage 2 therapy may be to elevate the treatment site to a temperature within an ablative range, with or without the additional application of drug therapy or radiation from an external source, such as electron beam radiation therapy. The Stage 2 therapy may be repeated two or three or four or as many times as desired, with or without modifications, throughout the full course of clinical treatment for the patient. The timing between the Stage 1 and Stage 2 therapies, and, if utilized, any follow on therapeutic stages, will depend upon a variety of circumstances, including the nature of the cancer or other disease, the aggressiveness of the condition, the presence of failed previous therapeutic attempts, and other aspects of the patient's condition, as will be appreciated by those of skill in the art. Potential elapsed times between stages have been discussed elsewhere herein. Since the implant may be permanently left in place, the opportunity always exists for at least follow on thermal therapy, either in the hyperthermia or the ablative temperature ranges.

The desired temperature for the initial thermal therapy, as well as follow on thermal therapies, may be determined by the physician, in accordance with the present invention. This is accomplished by selecting a ferromagnetic material having a Curie point which is equal to or exceeds the highest desired therapeutic temperature. Thus, a ferromagnetic material having a Curie point in the temperature range of 50° C. to about 100° C., often with the range of from about 60° C. to about 80° C., may be selected. In one exemplary implant, the Curie point of the ferromagnetic alloy is about 70° C. This implant may be readily heated to approximately 70° C. by exposure to an oscillating magnetic field as has been discussed previously herein. In accordance with the present invention, the same implant may subsequently or previously be heated to a lower temperature, by altering the parameters of the magnetic field. For example, by pulsing the magnetic field between on and off states, the tissue surrounding the implant may be maintained at a temperature below the Curie point. This is due to the natural thermal dissipation which occurs in living tissue, as heat is absorbed by surrounding tissue and fluids, and also carried away by localized microcirculation and other factors. Thus, by selecting a pulse duration and a spacing between pulses, taking into account the thermal relaxation or thermal dissipation rates of the surrounding tissue, the pulse width and pulse spacing can be optimized to maintain a predetermined temperature.

In a further aspect of the present invention, another method of treating a patient is provided. A patient having a previously positioned implant is identified, such as an implant having a fully decayed isotope thereon. The implant is activated to deliver a therapeutic modality to the patient. In certain embodiments, the patient's condition will be assessed prior to activating the implant.

The implant preferably comprises a ferromagnetic core, such as a palladium-cobalt alloy, having a Curie point temperature between about 41.5C and 100C. The implant also is preferably coated with an isotope, such as Pd-103, among others. The isotope layer preferably covers at least 60%, or more preferably at least 85%, or most preferably at least 95% of the outside surface of the core. In one embodiment, the isotope layer covers substantially the entire surface of the implant. The isotope layer may also be covered, at least in part by an outer layer. In one embodiment, outer layer comprises a metal, such as palladium, with a thickness from about 0.1 micron to about 20 microns. Alternatively, the outer layer may comprise a polymer.

The activating step preferably comprises heating the implant to a temperature of about 40C to about 100C. The implant is preferably activated by exposing the implant to a magnetic field to deliver heat to the patient. An external oscillating magnetic field, preferably with a maximum flux density between 25 gauss and 100 gauss and a frequency between 25 kHz and 200 kHz, is supplied, which acts upon the exemplary Pd—Co alloy elements. Pd—Co heats up under the influence of the oscillating magnetic field until the Curie point temperature is reached. The implant may be exposed to an oscillating magnetic field in a plurality of sessions over a course of treatment.

The embodiments described herein have several advantages over the prior art. As discussed above, solid radioactive pellets generally have greater source strength than radioactive coatings or implanted layers. Also, in the current invention, a material for encapsulating the radioactive pellets can be chosen, which allows transmission of ionizing radiation from the radioactive pellet to the surrounding tissue with minimal attenuation. At the same time, heating of tumorous tissue is maximized because the ferromagnetic heating elements are made of solid material with no encapsulation. Manufacturing is relatively simple, and seeds can be economically produced.

Embodiments of the present invention which include the uniform isotope layer extending around the periphery of a seed or rod, and also around the ends of the seed or rod, appear to deliver superior dosing characteristics compared to other forms of radioactive seeds. This is true whether or not the seed or rod is additionally capable of generating heat through the ferromagnetic material utilized in certain aspects of the invention.

The dosimetric characteristics in water of the brachytherapy Pd¹⁰³ source described below have been theoretically determined by using the MCNP Monte Carlo code [1]. Dose rate constant, radial dose function and anisotropy functions of the source have been obtained following the TG-43 recommendations [2]. In general, implants produced in accordance with the layered isotope aspect of the present invention appear to exhibit excellent anisotropy characteristics.

A Monte Carlo N-particle Transport Code (MCNP) [1] was used to calculate the dose rate distribution in water, Solid Water™ and dry air about an implant in accordance with the present invention (the “test implant”). The test implant consists of a cylindrical core (which is 93.35% palladium and 6.65% cobalt), 10 mm long and 1 mm diameter, uniformly coated around its sidewall and end by 50 nm radioactive ¹⁰³Pd. The outer shell is 7 microns of non-radioactive palladium. In order to validate the Monte Carlo simulation, a similar method has been applied for the dose rate calculation around previously published sources. An excellent agreement has been reached.

The dose rate constant is defined as the dose rate to water at a distance of 1 cm on the transverse axis of a unite air kerma strength source in a water phantom, namely,

Λ=D(r₀,θ₀)/SK (1)

Where, D(r₀, θ₀) is the dose rate at the reference point of (r₀, θ₀) along the transverse bisector of the source. The most commonly used reference point is r₀=1 cm θ₀=90 degree. Sₖ is the air-kerma strength of the source, which was calculated by interpolation of the air kerma rate at distances ranging from 0.5 to 25 cm. Graph 1 below shows a plot of air-kerma*r²⁰ graphed as a function of distance. This data indicated that the variation of the air kerma rate is less than 0.5% at distances larger than 5 cm. Therefore, the value of the air kerma rate at 10 cm distance was used to determine the air-kerma strength at 1 cm using the inverse square law. A ratio of the calculated dose rate in water at the reference point to the calculated air kerma strength was used to determine the dose rate constant of the implant. These results had indicated a dose rate constant of 0.650 cGy h⁻¹U⁻² for calculated air kerma strength was used to determine the dose rate constant of the implants in water.

[image]

The radial dose function, g(r), accounts for the effects of absorption and scatter in the medium along the transverse axis of the source,

$$g ( r ) = \frac{\dot{D} ( r , {θ}_{0} ) \cdot G ( {r}_{0} , π / 2 )}{D ( {r}_{0} , {θ}_{0} ) \cdot G ( r , π / 2 )} \qquad ( 2 )$$

G(r, θ₀) is the geometry function of the source, which is defined in TG43 report. An active length of 10 mm was used to determine the G(r, θ). Thus, we can obtain the radial dose function g(r).

[image]

TABLE I
Radial Dose Function

| Distance (cm) | Implant (water) |
|---|---|
| 0.5 | 1.226 |
| 1.0 | 1.000 |
| 1.5 | 0.789 |
| 2.0 | 0.609 |
| 2.5 | 0.465 |
| 3.0 | 0.351 |
| 3.5 | 0.263 |
| 4.0 | 0.201 |
| 4.5 | 0.151 |
| 5.0 | 0.113 |
| 6.0 | 0.063 |
| 7.0 | 0.036 |
| 8.0 | 0.020 |
| 9.0 | 0.011 |
| 10.0 | 0.006 |

Table I shows the values of the radial dose function of the test implant in water. Graph 2 is the Monte Carlo simulated radial dose function for the test implant.

The anisotropy function of a brachytherapy source accounts for the self-absorption of the distribution around the source, including the effects of absorption and scatter in the medium. It is defined as,

$$F ( r , θ ) = \frac{D ( r , θ ) G ( r , {θ}_{0} )}{D ( r , {θ}_{0} ) G ( r , θ )}$$

Anisotropy functions of the test implant in water were calculated at the radial distances of 2, 3, 5 and 7 cm. These calculations were performed at the 10-degree interval, from 0 to 360 degrees. The final results were expressed in one quadrant as shown in Graph 3. From the anisotropy function, the anisotropy factors, φ(r), and anisotropy constant, \overline{φ}ₐₙ, for the test implant have been determined following the AAPM TG-43 formalism. These results indicate that the anisotropy constant (inverse distance square weighted) of the new source is 0.99 in water.

[image]

TABLE II
Dose rate anisotropy function in water.

| Angle (degree) | 2 cm | 3 cm | 5 cm | 7 cm |
|---|---|---|---|---|
| 0 | 0.404 | 0.440 | 0.508 | 0.501 |
| 10 | 0.628 | 0.637 | 0.659 | 0.629 |
| 20 | 0.876 | 0.832 | 0.793 | 0.812 |
| 30 | 0.939 | 0.920 | 0.908 | 0.880 |
| 40 | 0.985 | 0.979 | 0.945 | 0.928 |
| 50 | 1.008 | 1.007 | 0.986 | 0.957 |
| 60 | 1.020 | 1.021 | 1.009 | 0.998 |
| 70 | 1.007 | 1.011 | 0.996 | 0.986 |
| 80 | 0.998 | 0.991 | 0.986 | 0.983 |
| 90 | 1.000 | 1.000 | 1.000 | 1.000 |
| Φ(r) | 1.002 | 0.983 | 0.961 | 0.947 |

| | |
|---|---|
| \overline{Φ}ₐₙ | 0.99 |

### REFERENCE

[1] RSICC Computer code collection “Monte Carlo N-particle Transport Code System”. Los Alamos National Laboratory, Los Alamos, N. Mex.

[2] Ravinder Nath, Lowell L.Anderson,Gary Luxton, Keith A. Weaver, Jeffrey F. Williamson and Ali S.Meigooni, “Dosimetry of interstitial brachytherapy source: Recommendations of the AAPM Radiation therapy committee task group No.43” Med. Phys.22, 209-234 1995)

[3] A. S. Meigooni, K. Sowards and M.Soldano, “Dosimetric Characteristics of the brachytherapy source”, Med.Phys.27, 1093-1100(2000)

[4] Sou-Tung Chiu-Tao and Lowell L.Anderson, “Thermoluminescent dosimetry for 103Pd seeds (model 200) in solid water phantom”, Med. Phys.18, 449-452(1991)

[5] R.Ewallace and J.Jfan “Dosimetric characterization of a new ¹⁰³Pd brachytherapy 65-2470(1999)

[6] A.Meigooni, K.Sowards and M.Soldano, “Dosimetric characteristics of the Intersource ¹⁰³palladium brachytherapy source”, Med.Phys.27, 1093-1100(2000)

[7] Keith Weaver, “Anisotropy functions for ¹²⁵I and 103Pd sources”, Med.Phys.25, 2271-2278 (1998)

This invention has been described herein in considerable detail to provide those skilled in the art with information relevant to apply the novel principles and to construct and use such specialized components as are required. However, it is to be understood that the invention can be carried out by different equipment, materials and devices, and that various modifications, both as to the equipment and operating procedures, can be accomplished without departing from the scope of the invention itself.

What is claimed is:

1. A method of treating a patient, comprising the steps of:
positioning an implant within a patient;
delivering a first therapeutic modality from the implant to the patient; and
activating the implant following the delivering a first therapeutic modality step to deliver a second therapeutic modality to the patient.

2. A method of treating a patient as in claim 1, wherein the delivering a first therapeutic modality step comprises delivering radiation to the patient.

3. A method of treating a patient as in claim 1, wherein the activating step comprises exposing the implant to a magnetic field.

4. A method of treating a patient as in claim 3, wherein the delivering a first therapeutic modality step is accomplished over a delivery period.

5. A method of treating a patient as in claim 4, comprising a delay between the delivery period and the activating step.

6. A method of treating a patient as in claim 5, wherein the delay is at least three months.

7. A method of treating a patient as in claim 5, wherein the delay is at least six months.

8. A method of treating a patient as in claim 1, wherein the delivering a first therapeutic modality step comprises delivering a drug to the patient.

9. A method of treating a patient as in claim 8, wherein the drug comprises an anti inflammatory agent.

10. A method of treating a patient as in claim 8, wherein the drug comprises an anti proliferative agent.

11. A method of treating a patient as in claim 8, wherein the drug comprises an antibiotic.

12. A method of treating a patient as in claim 8, wherein the implant comprises an endoluminal prosthesis.

13. A method of treating a patient as in claim 12, wherein the implant comprises a stent.

14. A multiple stage method of treating tissue, comprising the steps of:
positioning an implant into tissue to be treated;
delivering a dose of radiation from the implant to the tissue;
exposing the implant to a magnetic field; and
delivering heat to the tissue in response to the exposing step;
wherein the exposing step is accomplished following the end of the delivering a dose step.

15. A method as in claim 14, wherein the exposing step is performed at least 5 days after the end of the delivering a dose step.

16. A method as in claim 14, additionally comprising the step of delivering heat to the tissue during the delivering a dose of radiation step.

17. A method as in claim 16, wherein the field has a maximum flux density between about 25 gauss and about 100 gauss.

18. A method as in claim 16, wherein the field oscillates within the range of from about 25 kHz to about 200 kHz.

19. A method of treating a patient, comprising the steps of:
identifying a patient having a previously positioned implant therein;
activating the implant to deliver a therapeutic modality to the patient.

20. A method as in claim 19, wherein the implant comprises a ferromagnetic core.

21. A method as in claim 20, wherein the ferromagnetic core comprises a palladium-cobalt alloy.

22. A method as in claim 20 wherein the core exhibits a Curie point in a therapeutic range between about 41.5 C and about 100 C.

23. A method as in claim 20, wherein the implant further comprises a decay product of an isotope layer.

24. A method as in claim 20, wherein the isotope comprises Pd-103.

25. A method as in claim 20, wherein the isotope covers within the range of from about 35% to about 65% of the outside surface of the core.

26. A method as in claim 20, wherein the isotope covers at least about 85% of the outside surface of the core.

27. A method as in claim 20, wherein the isotope covers at least about 95% of the outside surface of the core.

28. A method as in claim 20, comprising an outer layer over the isotope layer.

29. A method as in claim 28, wherein the outer layer comprises a polymer.

30. A method as in claim 28, wherein the outer layer comprises a metal.

31. A method as in claim 30, wherein the outer layer comprises palladium.

32. A method as in claim 31, wherein the outer layer has a thickness from about 0.1 micron to about 20 microns.

33. A method as in claim 20, wherein the activating step causes the seed to heat to a temperature within the range of about 40C to about 100C.

34. A method of treating a patient as in claim 19, wherein the identifying step comprises identifying an implant having a fully decayed isotope thereon.

35. A method of treating a patient as in claim 19, wherein the activating step comprises exposing the implant to a magnetic field.

36. A method of treating a patient as in claim 19, wherein the activating step comprises activating the device to deliver heat to the patient.

37. The method of claim 19, wherein the activating step comprises exposing the implant to an oscillating magnetic field having a maximum flux density between about 25 gauss and about 100 gauss.

38. The method of claim 37, wherein the oscillating magnetic field has a range of frequency from about 25 kHz to 200 kHz.

39. The method of claim 19 wherein the implant is exposed to an oscillating magnetic field in a plurality of sessions over a course of treatment.

40. The method of claim 19, further comprising the step of clinically assessing the condition of the patient in the vicinity of the implant prior to the activating step.

41. The method of claim 19, wherein the identifying a patient step comprises identifying a patient having a previously positioned brachytherapy device therein.

42. The method of claim 41, wherein the activating the implant step comprises activating the implant to deliver heat in an ablation temperature range.

43. The method of claim 42, wherein the temperature is at least about 70° C.

44. The method of claim 19, wherein the activating step is only accomplished if the previously positioned implant failed to achieve its desired clinical result.

45. The method of claim 44, wherein the previous implant is deemed to have failed if the patient's PSA is at least about 0.75 ng/ml/year.

46. A method of heating tissue to at least two distinct temperatures from a single source, comprising the steps of:
identifying a medical device in contact with tissue, the device comprising a ferromagnetic material;
heating the tissue to a first temperature in response to exposing the device to a magnetic field under a first set of conditions; and thereafter heating the tissue to a second temperature in response to exposing the device to a magnetic field under a second set of conditions.

47. A method of heating tissue as in claim 46, wherein the first temperature is a hyperthermia temperature.

48. A method of heating tissue as in claim 46, wherein the second temperature is an ablative temperature.

49. A method of heating tissue as in claim 46, wherein the first temperature is a hyperthermia temperature, and additionally comprising the step of expressing radiation form the device to the tissue.

50. A method of heating tissue as in claim 46, wherein at least one of the first and second temperatures is approximately the Curie point for the ferromagnetic material.

51. A method of heating tissue as in claim 50, wherein the other of the first and second temperatures is below the Curie point for the ferromagnetic material.

52. A method of heating tissue as in claim 50, wherein the other of the first and second temperatures is at least about 10 degrees C. below the Curie point for the ferromagnetic material.

53. A method of heating tissue as in claim 51, wherein the other of the first and second temperatures is achieved by pulsing the magnetic field.

54. A method of heating a medical implant to a temperature below the Curie point for a ferromagnetic material carried by the implant, comprising exposing the implant to an oscillating magnetic field, and pulsing the magnetic field to cooperate with the dissipation of heat from the implant to maintain the implant at a temperature that is below the Curie point.

55. A method of achieving a preselected temperature in tissue surrounding an implanted ferromagnetic material having a Curie point, comprising the steps of exposing the material to an oscillating magnetic field, and pulsing the field to achieve the desired temperature in the tissue, wherein the desired temperature is below the Curie point.

56. A method of treating a patient, comprising the steps of:
exposing the patient to a magnetic field to generate temperatures in the hyperthermia range at a treatment site within the patient; and
exposing the patient to a magnetic field to generate temperatures in the ablative range at the treatment site within the patient.

57. A method of treating a patient as in claim 56, additionally comprising the step of delivering radiation to the treatment site while the treatment site is at a temperature in the hyperthermia range.
